(19) United States
(12) Patent Application Publication
(10) Pub. No.: US 2014/0155943 A1
(43) Pub. Date: Jun. 5, 2014

(54) BONE IMPLANT

(71) Applicant: Henrik Andersen, Skibby (DK)

(72) Inventor: Henrik Andersen, Skibby (DK)

(73) Assignee: Elos Medtech Pinol A/S

(21) Appl. No.: 14/130,437

(22) PCT Filed: Jun. 29, 2012
(86) PCT No.: PCT/DK2012/050233

§ 371 Date: Dec. 31, 2013

(30) Foreign Application Priority Data
Jul. 1, 2011 (EP) 11172418.3

(51) Int. Cl. A61B 17/86

(52) U.S. Cl. CPC A61B 17/8625
USPC 606/312

(57) ABSTRACT

The present invention relates to a bone implant device, comprising a body having a first part and an opposite second part, the first part and an opposite second part defining a longitudinal axis, the first part including an external thread extending from a cylindrical part to the distal end of the body, the external thread comprising a first thread part having a first radius measured from the longitudinal axis and a second thread part having a second radius measured from the longitudinal axis. The first radius is larger than the second radius, the first thread part is a cutting part, and the second thread part is a contact surface. The first part may include one or more cavities for storing bone fragments while the bone implant is in use.

### FIELD OF THE INVENTION

The present invention relates to a bone implant device.

### BACKGROUND OF THE INVENTION

There are several situations where a bone implant is needed. Below is listed some situations where a bone implant is usually used.

When a person has a fractured bone there is a need for bringing two or more bone parts together so that the bone may repair the fracture.

When a person needs a tooth implant mounted in the jaw-bone, there is a need for an implant device to hold the tooth implant.

When a person has a hearing impediment there may in some situations be a need for mounting a hearing directly to the bone of the skull. For mounting such a hearing aid there is a need for an implantable device for attaching the hearing aid to the skull. The implant should not damage the bone structure in a way that will lead to necrotise of the surrounding bone material. Usually a relatively large hole is predrilled before the implant is inserted. Threads on the implant will cause the implant to be partly fastened in the bone, and the bone will repair itself. The implant will most likely be subject to oseointegration.

Some geometry of previous implants, in all applications mentioned above, has shown to cause compression and/or heating of the bone material thus damaging the bone material; sometimes to such a degree that the bone dies. The friction, compression and the energy exerted when inserting the bone implant have shown to cause damage to the bone, also with subsequent bone necrosis. One solution to these problems has been to remove a major part of the thread and the body of the implant device. This however leads to other problems such as a low initial/primary stability as the implant device will not have sufficient grip in the bone, further the relative low amount of bone material will have to grow back which gives the patient a long recovery period.

Damage to bone due to the insertion of a bone implant will cause longer healing times and thus more inconveniences to the users. Also, necrotic bone material may be a serious problem, especially in skull bone.

Thus there is a need for an implant device that at least alleviates the above-identified problems.

It is one object of the present invention to provide an implant device where the surrounding bone heals faster than when using other implant devices.

Further it is an object to provide an implant having a high initial/primary stability, i.e. a device that is not inclined to budge or move relative to the bone that it has been inserted into. It is a further object of the present invention to provide an alternative to the prior art.

### SUMMARY OF THE INVENTION

As mentioned the present invention relates to a bone implant device with a thread, such as a screw-shaped anchoring element for permanent or temporarily anchorage in bone.

The bone implant device is in one embodiment suitable for joining bone parts. Such as two or more bone parts, e.g. part of humerus, femur or other bone.

The bone implant device is in one embodiment suitable for supporting an artificial tooth, a hearing aid devices or extraoral prostheses in the form of ear and orbital prostheses in the skull bone.

The implant device according to the present invention is in one embodiment suitable for implants in the jaw bone for supporting artificial teeth.

However, the invention is not limited to these specific applications, and may be used in connection with other types of devices to be anchored in bone

Thus, the above described object and several other objects are intended to be obtained in a first aspect of the invention by providing a bone implant device comprising a body having a first part and an opposite second part, the first part and an opposite second part defining a longitudinal axis, the first part including an external thread extending from a cylindrical part to the distal end of the body, the external thread comprising a first thread part having a first radius measured from the longitudinal axis and a second thread part having a second radius measured from the longitudinal axis, and the first radius is larger than the second radius, wherein the first thread part is a cutting part and the second thread part is a contact surface, the first part having one or more cavities for storing bone fragments while the bone implant is in use. The external thread may thus comprise a number of first and second thread parts arranged along the length of the external thread. One or more cavities are formed in the first part and in the cavities bone fragment are collected as the implant is inserted. Further details described through-out the present specification also applies to this aspect. At the end of the cavity farthest from the distal end of the implant the cavity may be closed off by the cylindrical part so that bone fragments will be kept near the implant and not transported away from the implant. The one or more cavities are preferably shaped so that they extend along the longitudinal length of the bone implant. The cavity or cavities may be

The present invention may relate to a bone implant device comprising a body having a first part and an opposite second part, the first part and an opposite second part defining a longitudinal axis, the first part including an external thread, and the external thread comprising a first thread part having a first radius measured from the longitudinal axis and a second thread part having a second radius measured from the longitudinal axis, and the first radius is larger than the second radius, wherein the first thread part is a cutting part and the second thread part is a contact surface.

When inserting an implant device in bone, a hole is predrilled. The hole will have a diameter slightly smaller than the diameter of part of the implant device that is to be inserted into the bone. The implant device is then screwed into the bone, and the threaded part will cut the bone so that the implant device is securely mounted in the bone.

Compared to the geometry of the prior implant devices the implant device according to the present invention will have a minimum of gap between the bone and the implant device as there are no large openings are present which need to be filled with new bone material. As there is a relatively large contact surface between the implant device and the bone. The healing process will be shorter and a high initial/primary stability is gained.

Following the path of the tread from one point and towards the end of the thread a ‘first’ first thread part will have a smaller radius than a following neighbouring first tread part so that the overall radius grow thereby gradually cutting deeper into the bone material. Thus in two neighbouring sections of the thread the first section will have a first thread part having a first radius being smaller than the second sections corresponding first radius. Thereby a ‘jump’ in radius is established.

Advantageously the second part of the body may include an external thread. Such an implant device will thus have an external thread along the entire body of the device. This may be advantageous when mounting the device in an area with a relative large amount of dense bone material or when it is difficult to determine the exact amount of dense bone that the device may be attached in.

It is contemplated that the body and the external thread will be embedded in the bone structure. When mounting the implant device a hole is predrilled in the bone so that the implant device is not required to remove or displace bone in order for the implant device to be inserted into the bone. The first and second opposite parts are the two ends of the implant device, where one part carries the external thread and the other part is used for engaging a tool used to insert the implant device into bone. The structure or geometry of the implant device has some similarities with that of screws for wood, i.e. a body defining a longitudinal axis in the length of the device. In an embodiment where the implant device is intended for implantation in skull bone of a human, the overall length of the implant device is short relative to the width of the implant device, whereas in an embodiment where the implant device is intended to be used in jaw bone the overall length of the implant device relative to the width is larger.

It is contemplated that having neighbouring areas of the external thread having different radii, i.e. one area where the radius is one size and another, adjacent, area where the radius is different, will overall provide a device having the desired properties, including but not limited to low insertion torque required and high initial/primary stability. This is at least partly achieved by the step in diameter or radius as described above.

The external thread have, as mentioned, two functions, one function is cutting the bone while the device is being mounted and also to support the implant device and provide a large initial/primary stability.

Advantageously the first thread part, i.e. the cutting part, leads the second thread part when the implant device is inserted into bone. This allows cutting the bone without causing too much compression, i.e. to a level where the bone is damaged by the compression. Compression damage may lead to bone necrotise which in turn lead to the implant device not being integrated in and supported by the bone.

The step in diameter or radius as described above may advantageously include a transition part formed between the first thread part and the second thread part and the transition part defines a smooth transition. The transition part is contemplated to allow the area which supports the implant device to be relatively large.

The step in diameter or radius as described above may advantageously include a transition part formed between the first thread part and the second thread part and the transition part defines a discontinuous transition. This is contemplated to allow establishment of a smaller area of the supporting part compared to the continuous transition as described above. The optimal size of the support area may be chosen dependent on a specific application, e.g. one may be more suitable for implants intended for jaw bone and one optimal size may be more suitable for implants intended for skull bone. The implant device may be used for bones in arms, legs, feet, hands or any other such bones. The implant device may be used for straightening the backs of people suffering from osteoporosis where a number of devices are mounted in the vertebra column of a person which devices are then connected to a bar or the like. The implant device could also be used for animals.

Advantageously the bone implant device may comprise one or more cavities at the first part of the bone implant device. Cavity or recess or depression or cut-out area reduce the area by which the implant device is supported in the bone, but also reduces the area causing friction and compression but as mentioned elsewhere too large cavities are not desired. A cavity or depression or cut-out area will be filled by bone fragments during the insertion process. By designing the cavity optimally the size of the cavity will not be so large that the surrounding bone material needs to grow for an extended period of time. The bone fragments is contemplated to allow faster healing of the bone as the fragments will be reintegrated with the bone in the intended part of the body.

Advantageously the first thread part is adjacent the at least one cavity. This allows bone fragments from the cutting of the bone implant to be collected at these cavities.

Advantageously the first part defines a tapered geometry. As the bone implant is self-cutting, this geometry is contemplated to be most advantageous as it allow the bone implant to cut into bone material that has not been pre-threaded.

Advantageously the first part includes a substantially flat base portion and the external thread extends along the first part to the substantially flat base portion. This ensures that the bone implant is sufficiently cut into the bone and the substantially flat base portion ensures that the bone implant does not extend unnecessarily deep into the bone. Alternatively the first part may include a smooth surface in the form of part of a sphere so that in case the bone implant is inserted too far into the bone, e.g. when mounting in skull bone, there is a reduced risk of damage to the brain caused be sharp edges on the bone implant device.

Advantageously the first part may by partly or completely hollow with an opening at the end of the implant device. This may allow bone to grow into the opening and thus integrate better with the implant device, either with or without oseointegration.

Advantageously the bone implant device comprises a flange disposed at the second part arranged so that the flange defines a plane substantially transvers to the longitudinal axis. The flange allows the bone implant device to be inserted to a certain maximum depth.

Advantageously the bone implant device may have a threaded part at both ends, so as to join two bone parts. One of the threaded parts may then have a smaller thread than the other. The two parts may then be inserted in two bone parts that are to be joined. The two threaded parts may be joined by a part not having an external thread. Alternatively the entire body of the bone implant device may be covered by thread.

Advantageously the bone implant device may be configured to connect to an ear-prosthesis, an orbital prosthesis, a nose prosthesis, a set of dentures or a single prosthetic tooth, a hearing aid or a bone anchored hearing device.

Advantageously the bone implant device may be configured to establish fusing of two bones. This may be achieved by the bone implant device comprising a third part opposite the second parts, and the third part including an external thread, the external thread of the third part comprising a third thread part having a third radius measured from the longitudinal axis and a fourth thread part having a fourth radius measured from the longitudinal axis, and the third radius is larger than the fourth radius, wherein the third thread part is a cutting part and the fourth thread part is a contact surface. As described above in relation to the first part of the bone implant the external thread at the third part may extend from a cylindrical part. Further the third part may include one or more cavities for collecting and storing bone fragments, as described in relation to the first part elsewhere.

Advantageously the threaded part at the first part may have a first pitch and the threaded part at the third part may have a second pitch being different from the first pitch. This may allow the two parts to be drawn together.

Advantageously the bone implant device may further comprise a hollow space having an opening at the distal end of the first part. The hollow space may be a cavity, i.e. a hole in the body with an opening so as to allow bone to grow into the hollow space. This is contemplated to improve the stability of the implant device in the bone.

Advantageously the bone implant device is made from stainless steel or titanium. The stainless steel is preferably approved for medical use.

The invention is particularly, but not exclusively, advantageous for obtaining an implant device having a high initial/primary stability. Further the bone implant device has a low insertion torque.

These and other aspects of the invention will be apparent from and elucidated with reference to the embodiments described hereinafter.

### BRIEF DESCRIPTION OF THE FIGURES

The invention will now be described in more detail with regard to the accompanying figures. The figures show one way of implementing the present invention and is not to be construed as being limiting to other possible embodiments falling within the scope of the attached claim set.

FIG. 1 schematically illustrate a perspective view of an embodiment of an implant according to the present invention,

FIG. 2 schematically illustrate a side-view of an embodiment of an implant according to the present invention,

FIG. 3 schematically illustrate a bottom-view of an embodiment of an implant according to the present invention,

FIG. 4 schematically illustrate a cut-through view of an embodiment of an implant according to the present invention,

FIG. 5 schematically illustrate a partial view of an external thread of an embodiment of an implant according to the present invention,

FIG. 6 is a schematic graph illustrating torque vs. insertion depth,

FIG. 7 is a schematic graph illustrating radius vs. thread length,

FIG. 8 is a schematic graph illustrating radius vs. thread length,

FIG. 9 is a schematic illustration of an implant device with threaded parts at both ends,

FIG. 10 is a schematic illustration of an implant device having a relatively small threaded part,

FIGS. 11 and 12 are schematic illustration of radii of other types of implants.

### DETAILED DESCRIPTION OF AN EMBODIMENT

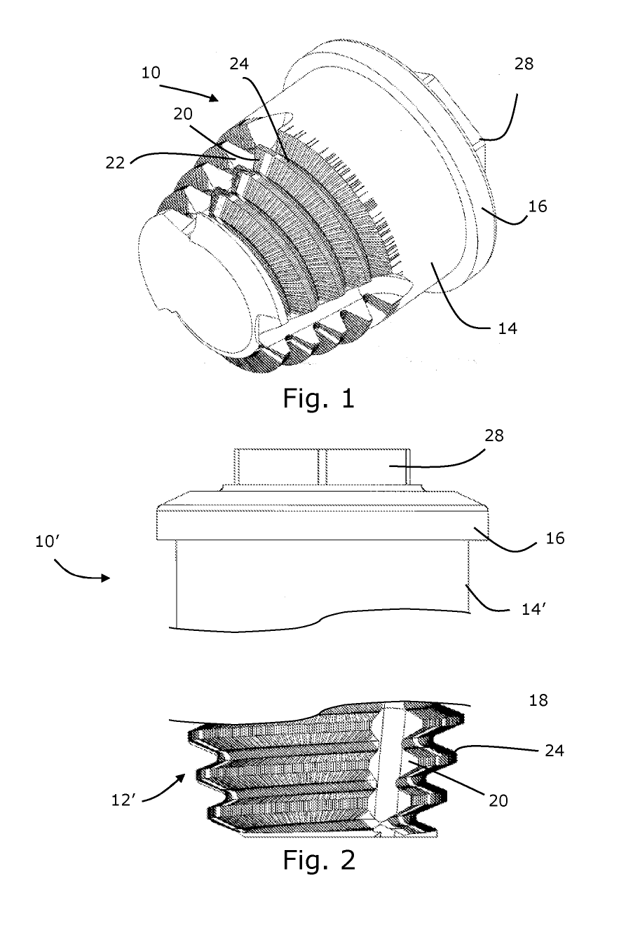

FIG. 1 is a schematic illustration of a bone implant device 10 according to the present invention.

The bone implant device 10 comprises a body having a first part 12 and an opposite second part 14. The first part 12 and the opposite second part 14 define a longitudinal axis, not illustrated. The first part 12 includes an external thread 18. The external thread 18 comprises a first thread part 20 having a first radius measured from the longitudinal axis and a second thread part 22 having a second radius measured from the longitudinal axis. The first radius is larger than the second radius. The first thread part 20 is a cutting part and the second thread part is a contact surface. The contact surface rests against the bone when the implant device 10 has been inserted. When inserting the implant device 10 the contact surface will slide over a part of the bone where the first part 20, i.e. the cutting part, has left a space. By reducing the radius the actual contact area during insertion is reduced which also reduces heat generation due to friction. The thread design allow the implant device is be inserted into bone without damaging the bone, i.e. low compression of the bone, which again lead to lowered time for the bone to heal. The thread design also provides a minimum cut-out volume of the bone, which provides material for the thread to engage.

The implant device 10 includes a flange 16 which functions as a stop when the implant device 10 is inserted into the bone to ensure a maximum insertion depth. The flange 16 is disposed at the second part 14 and arranged so that the flange 16 defines a plane substantially transvers to the longitudinal axis. The longitudinal axis is normal to the plan defined by the flange 16. The plane defined by the flange 16 is flat in this embodiment. In other embodiments the flange may be formed so that it does not form a flat plane

When inserting the implant device 10 the person performing the operation may choose to stop before the flange 16 is in contact with the bone. In the embodiment illustrated the implant device 10 is intended for use in skull-bone and the main body 10 is shorter than 5 mm, in order not to go completely through the relatively thin skull bone, and it has a tapered portion with external threads.

The implant device 10 is made of titanium which has an ability to integrate into surrounding bone tissue, the so-called oseointegration as mentioned above. If the implant device is made from medically approved stainless steel the implant device is less likely to be subjected to oseointegration and may be more easily be removed from the bone.

The bone implant device 10 includes a transition part 24 being formed between the first thread part 20 and the second thread part 22 and the transition part 24 defines a smooth transition. This is also schematically illustrated by the graph in FIG. 7. The graph 34 illustrates radius of the thread as a function of thread length. A first section having a first thread part is illustrated at the line 52 defining the first ‘first radius’. A second section having a second thread part is illustrated at the line 56 defining the second ‘first radius’. Thereby the overall radius is increased along the thread. The lines 54 and 58 illustrate the respective ‘second radius’. Here it is seen that there is a jump in radius whereby a cutting edge is established and followed by a section where it is ensured that the bone is not compressed.

In other embodiments it is possible that the transition part formed between the first thread part and the second thread part defines a discontinuous transition. This is schematically illustrated by the graph in FIG. 8. The graph 36 illustrates radius of the thread as a function of thread length.

The bone implant device 10 further comprises a number of cavities; in FIG. 1 the cavities 26 and 28 are visible. The cavities 26 and 28 are positioned at the first part 12 of the bone implant device 10. The cavity, or depression or cut-out area, serves several purposes, one of which is to act as a collector of bone fragments originating from the first thread parts cutting the bone while the implant device is being inserted.

FIG. 2 is a schematic side view of a bone implant device 10′. The device 10′ is greatly similar to that illustrated in FIG. 1 so like elements are given like numerals. Here the second thread parts are seen opposite the cavity from the first thread parts. The geometry of the cavity is not important and the cavities do not need to have identical or similar geometries. Different geometries for the different cavities may be chosen. The bone implant device 10′ is illustrated with two wavy lines indicating that the length of the second part relative to the length of the first part may be longer or shorter depending on the specific intended purpose, e.g. a long implant device is needed for an application in jaw bone, a shorter implant device is needed for an application in skull bone.

The relative length of the part 12′ may be chosen depending on the application, e.g. a short implant device may have a relative long part 12′ to establish a good grip in a relatively thin bone part, whereas a relative long implant device for implantation in jaw bone may have a relative short part 12′ as a majority of the implant device will be embedded in spongy bone, i.e. not dense or compact bone.

In the embodiment illustrated in the figures, the first thread part 20 is adjacent a cavity on one side and a second thread part on the other side. This establishes a step in diameter from the second thread part on the one side of the cavity and the first thread part on the other side of the cavity.

Preferably the radius of the thread of the second thread part is constant over the section of the second thread part, but this is not required in all embodiments.

Overall the first part of the bone implant device 10 defines a tapered geometry.

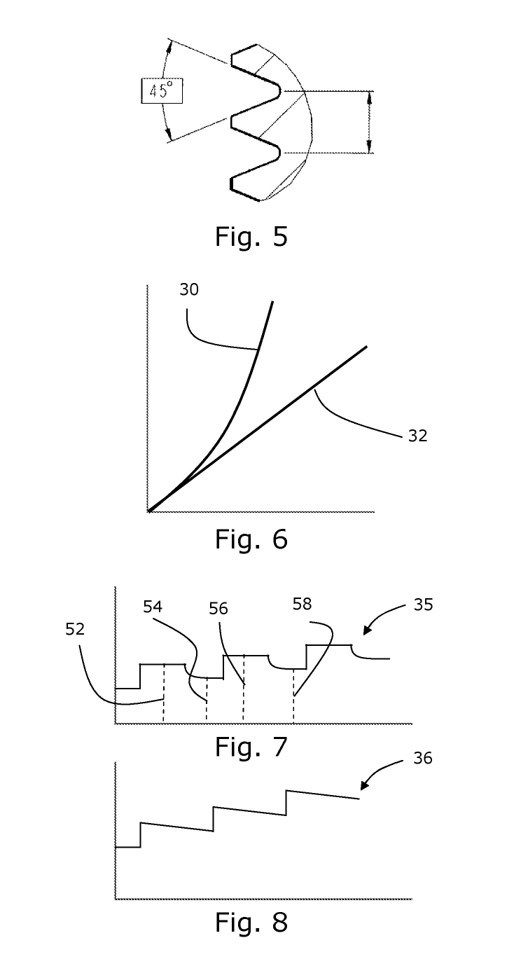

FIG. 5 schematically illustrates a part of the bone implant where it is seen that in this embodiment the angle defined by the groove between two thread ridges is around 45 degrees. This angle may depend on the tool used for cutting the thread. The distance between two succeeding grooves is approximately 0.6 mm.

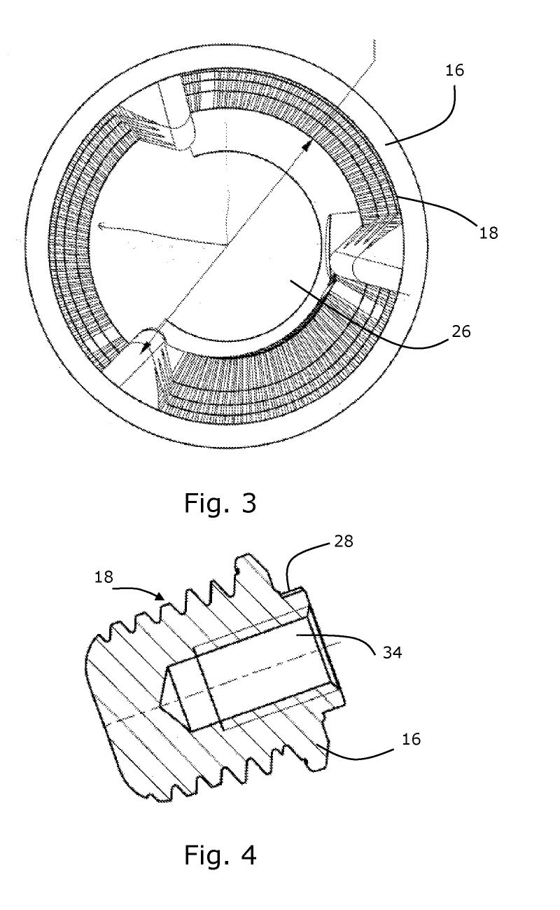

FIG. 3 is a schematic bottom view of the bone implant device 10. Here the substantially flat base portion 26 is seen. Three cavities are formed on the bone implant device 10. In other embodiments there may be more or less cavities. More than three cavities may be use full if the diameter of the bone implant device is relatively large and provided it is not desirable to simply increase the size of the individual cavity.

FIG. 4 is a schematic cut-through view of a bone implant device 10. The bone implant device 10 includes an opening 34 configured to receive an adapter for attaching a device, such as a hearing aid or of a type as mentioned above.

FIG. 6 is a schematic illustration of the torque required to insert a bone implant. The line 30 illustrates torque as a function of insertion depth required to insert an implant device not having the structure of the implant device according to the preset invention. The line 32 illustrates the torque as a function of insertion depth required to insert a bone implant device according to the present invention.

The bone implant device 10 includes a substantially flat base portion 26 and the external thread 18 extends along the first part 12 to the substantially flat base portion 26.

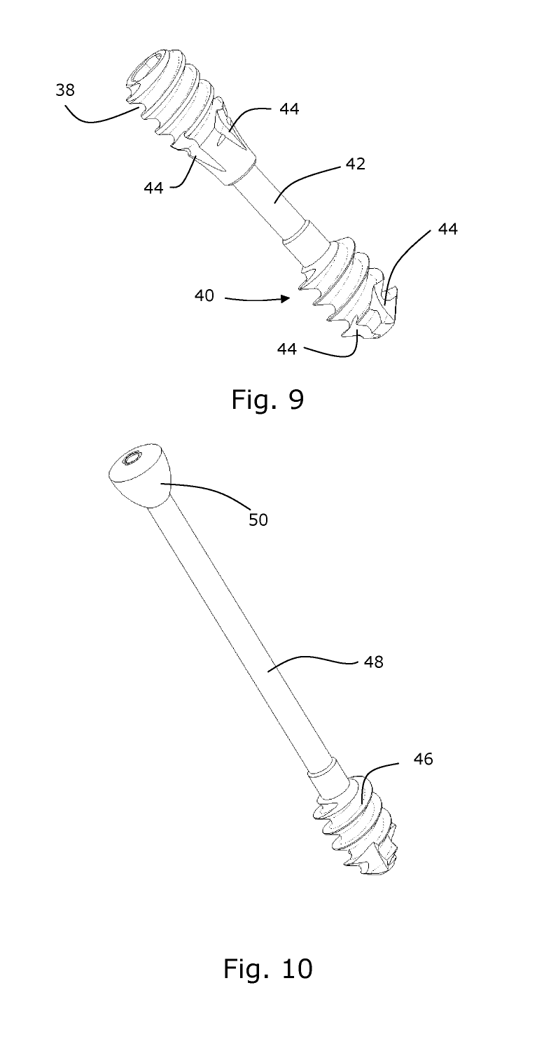

FIG. 9 is a schematic illustration of an implant device 10″ with threaded parts at both ends 38 and 40. The ends 38, 40 are connected via the part 42 which hold no external thread. The threads at the ends 38, 40 comprise cut-out areas 44 as described in relation to the embodiments above. The bone implant device 10″ may be used for joining two bone parts, where a difference in thread size between the two threads ensures that the bone parts are brought into contact.

FIG. 10 is a schematic illustration of an implant device 10′″ having a relatively small threaded part 46 relative to the overall length of the implant device 10′″. This embodiment is suitable for used in bone material where a large part of the bone is soft and thus not suitable for supporting the implant device 10′″, this includes e.g. jaw bone where the threaded part 46 is inserted in hard bone material while the part 48 extends through a lager soft bone material. At the end opposite the threaded part 46 a support 50 is provided. The support 50 may support one or more artificial teeth. The support 50 is configured for releasable mount of a device, e.g. the mentioned artificial tooth or the like.

In the embodiment illustrated in FIG. 1 the bone implant device 10 is configured to connect to a hearing aid or a bone anchored hearing conduction amplification device. By designing the overall length of the bone implant device, e.g. defining the length to be in the range 1-5 cm, it is possible to use the bone implant device for supporting an ear prosthesis, an orbital prosthesis, a nose prosthesis, a set of dentures or a single prosthetic tooth. The bone implant device will still provide the user with the benefits of low insertion torque, high initial/primary stability and other advantages. In still other embodiments, the bone implant device may be configured to establish fusing of a joint between two bones.

The external thread illustrated herein has a helical geometry. This geometry is contemplated to provide the best way of designing a thread.

Further the implant device 10 includes a tool engaging socket 28 in the form of an external hex. This external hex 28 establishes a tool engagement socket 28 for engaging a tool used for inserting the bone implant device in a bone.

The titanium used for the implant is in this embodiment of a type satisfying the ASTM F67 standard, namely unalloyed titanium for Surgical Implant Applications.

When a typical implant is operated into the bone of a patient, either the skull or jaw depending on the type of device the implant is to support, the bone surrounding the implant needs to regenerate. This means that the implant should not be loaded for an extended period of time as it may damage the reconstruction and thereby displacing the implant in relation to the intended orientation and/or damage the bone. By having a high initial/primary stability the bone implant is ensured not to be easily moved or displaced. Further the present implant device is contemplated to provide a fast recovery for the patient as the bone is minimally damaged during the insertion process.

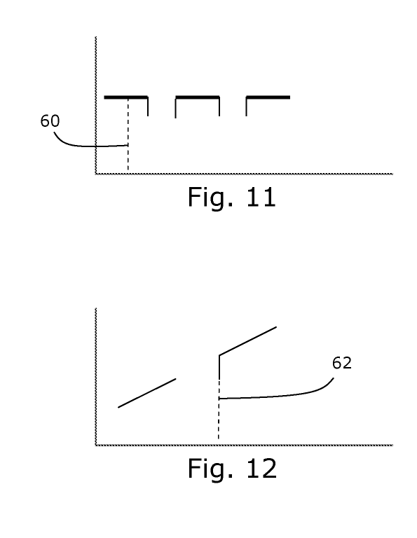

FIGS. 11 and 12 are schematic illustration of radii of thread for other types of implants. The radius of the thread in FIG. 11 is constant for a period and then the thread is removed in the areas where a piece of the body is cut-out for creating space. After that section the thread is returned to the same level. The punctured line 60 illustrated where the thread is returned. In order for this to work properly there is a need for removing a relatively large portion of the thread.

In FIG. 12 there is a constantly growing radius with cut-out areas giving a temporary removal of the thread to create a cutting edge and space. The punctured line 62 illustrated where the thread is returned. In order for this to work properly there is a need for removing a relatively large portion of the thread.

Although the present invention has been described in connection with the specified embodiments, it should not be construed as being in any way limited to the presented examples. The scope of the present invention is set out by the accompanying claim set. In the context of the claims, the terms “including” “includes” “comprising” or “comprises” do not exclude other possible elements or steps. Also, the mentioning of references such as “a” or “an” etc. should not be construed as excluding a plurality. Any use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

With reference to the drawings the following reference numerals are used:
10 Bone implant
12 First part
14 Second part
16 Flange
18 External thread
20 First thread part
22 Second thread part
24 Transition part
26 Base portion
28 Tool engagement socket
30 Line
32 Line
34 Opening
35 Graph
36 Graph
38 Thread
40 Thread
42 Part
44 Cut-out area
46 Thread
48 Part
50 Support
52 Line
54 Line
56 Line
58 Line

The present invention may be characterised by the following points

1. A bone implant device comprising:

a body having a first part and an opposite second part, the first part and an opposite second part defining a longitudinal axis,

the first part including an external thread,

the external thread comprising a first thread part having a first radius measured from the longitudinal axis and a second thread part having a second radius measured from the longitudinal axis, and the first radius is larger than the second radius, wherein the first thread part is a cutting part and the second thread part is a contact surface.

2. The bone implant device according to point 1, wherein a transition part is formed between the first thread part and the second thread part and the transition part defines a smooth transition.

3. The bone implant device according to point 1 wherein a transition part is formed between the first thread part and the second thread part and the transition part defines a discontinuous transition.

4. The bone implant device according to any one of the points 1-3, further comprising one or more cavities at the first part of the bone implant device.

5. The bone implant device according to point 4, wherein the first thread part is adjacent the at least one cavity.

6. The bone implant device according to any one of the points 1-5, wherein the first part defines a tapered geometry.

7. The bone implant device according to any one of the points 1-6, wherein the first part includes a substantially flat base portion and the external thread extends along the first part to the substantially flat base portion.

8. The bone implant device according to any one of the points 1-7, wherein the bone implant device is made from stainless steel and/or titanium.

9. The bone implant device according to any one of the points 1-8, wherein the bone implant device is configured to connect to an ear prosthesis, an orbital prosthesis, a nose prosthesis, a set of dentures or a single prosthetic tooth, a hearing aid or a bone anchored hearing conduction amplification device.

10. The bone implant device according to any one of the points 1-8, wherein the bone implant device is configured to establish fusing of a joint between two bones.

11. The bone implant device according to any one of the points 1-10, wherein the bone implant device comprises a third part opposite the second parts, and the third part including an external thread, the external thread of the third part comprising a third thread part having a third radius measured from the longitudinal axis and a fourth thread part having a fourth radius measured from the longitudinal axis, and the third radius is larger than the fourth radius, wherein the third thread part is a cutting part and the fourth thread part is a contact surface.

12. The bone implant device according to point 11, wherein the threaded part at the first part have a first pitch and the threaded part at the third part have a second pitch being different from the first pitch.

13. The bone implant device according to any one of the points 1-10, wherein the second part includes a tool engagement socket for engaging a tool used for inserting the bone implant device in a bone.

14. The bone implant device according to any one of the points 1-13, further comprising a hollow space having an opening at the distal end of the first part.

15. The bone implant device according to any one of the points 1-14, wherein the second part of the body includes an external thread.

1. A bone implant device comprising:
a body having a first part and an opposite second part, the first part and an opposite second part defining a longitudinal axis,
the first part including an external thread extending from a cylindrical part to the distal end of the body,
the external thread comprising a first thread part having a first radius measured from the longitudinal axis and a second thread part having a second radius measured from the longitudinal axis, and the first radius is larger than the second radius, wherein the first thread part is a cutting part and the second thread part is a contact surface, the first part including one or more cavities for storing bone fragments while the bone implant is in use.

2. The bone implant device according to claim 1, wherein a transition part is formed between the first thread part and the second thread part and the transition part defines a smooth transition.

3. The bone implant device according to claim 1 wherein a transition part is formed between the first thread part and the second thread part and the transition part defines a discontinuous transition.

4. The bone implant device according to claim 1, further comprising one or more cavities at the first part of the bone implant device.

5. The bone implant device according to claim 4, wherein the first thread part is adjacent the at least one cavity.

6. The bone implant device according to claim 1, wherein the first part defines a tapered geometry.

7. The bone implant device according to claim 1, wherein the first part includes a substantially flat base portion and the external thread extends along the first part to the substantially flat base portion.

8. The bone implant device according to claim 1, wherein the bone implant device is made from stainless steel and/or titanium.

9. The bone implant device according to claim 1, wherein the bone implant device is configured to connect to an ear prosthesis, an orbital prosthesis, a nose prosthesis, a set of dentures or a single prosthetic tooth, a hearing aid or a bone anchored hearing conduction amplification device.

10. The bone implant device according to claim 1, wherein the bone implant device is configured to establish fusing of a joint between two bones.

11. The bone implant device according to claim 1, wherein the bone implant device comprises a third part opposite the second parts, and the third part including an external thread, the external thread of the third part comprising a third thread part having a third radius measured from the longitudinal axis and a fourth thread part having a fourth radius measured from the longitudinal axis, and the third radius is larger than the fourth radius, wherein the third thread part is a cutting part and the fourth thread part is a contact surface, the third part including one or more cavities for storing bone fragments while the bone implant is in use.

12. The bone implant device according to claim 11, wherein the threaded part at the first part have a first pitch and the threaded part at the third part have a second pitch being different from the first pitch.

13. The bone implant device according to claim 1, wherein the second part includes a tool engagement socket for engaging a tool used for inserting the bone implant device in a bone.

14. The bone implant device according to claim 1, further comprising a hollow space having an opening at the distal end of the first part.

15. The bone implant device according to claim 1, wherein the second part of the body includes an external thread.
